(19) United States
(12) Patent Application Publication
(10) Pub. No.: US 2025/0001196 A1
(43) Pub. Date: Jan. 2, 2025

(54) AI DRIVEN PLATFORM TO USE INFRARED LIGHT TO TREAT INFLAMMATORY CYTOKINE STORMS

(71) Applicant: Photorevive, Inc., Lansdowne, PA (US)

(72) Inventor: Margaret Ahmad, Lansdowne, PA (US)

(21) Appl. No.: 18/753,878

(22) Filed: Jun. 25, 2024

Related U.S. Application Data
(60) Provisional application No. 63/510,527, filed on Jun. 27, 2023.

(51) Int. Cl. A61N 5/06; G16H 20/40

(52) U.S. Cl. CPC A61N 5/0613; G16H 20/40; A61N 2005/0627; A61N 2005/0659

(57) ABSTRACT

The invention provides in some aspects an apparatus for treatment of a patient for an inflammatory condition that includes a sensor to detect the presence and/or measure (e.g., quantity or quality) of each of one or more biomarkers of the patient, and that generates biomarker data indicative thereof. An illumination source that is in proximity of the patient applies a therapeutic dose of electromagnetic radiation to reduce the inflammation. A controller that is coupled to the sensor and the illumination source generates a dose control signal to effect application of the therapeutic dose by the illumination source. The controller determines an efficacy of applying such a dose by analyzing the biomarker data with an artificial intelligence (AI) engine and a machine learning (ML) model trained with training data indicative of modulation of the one or more biomarkers in response to dosing of such electromagnetic radiation.

### BACKGROUND OF THE INVENTION

This application claims the benefit of U.S. Patent Application Ser. No. 63/510,527, filed Jun. 27, 2023, entitled, “AI Driven Platform to Use Infrared Light to Treat, Inflammatory Cytokine Storms,” the teachings of which are incorporated herein by reference.

The invention relates to photobiomodulation therapy-based medical apparatus and methods of treatment. It has application, by way of non-limiting example, in treating life-threatening inflammation caused by excessive production of pro-inflammatory cytokines (cytokine storms).

Photobiomodulation therapy (PBMT) is the controlled application of light photons—typically infrared and visible light—for medically therapeutic purposes including for treating injury, disease, pain and immune system distress. PBMT is a type of phototherapy that broadly refers to the therapeutic application of light by means of a variety of therapies and treatment regimens.

Life-threatening inflammation or sepsis is the body's extreme response to an infection through the uncontrolled escalation of the inflammatory immune response. Under normal conditions, inflammation causes secretion of cellular factors, known as cytokines, which trigger defense mechanisms that destroy and remove damaged cells and pathogens. However, in some cases the inflammation persists beyond the end of the primary infection, particularly after acute episodes. Under conditions of ongoing inflammation, healthy cells become destroyed instead of diseased ones, which in turn causes release of additional toxins that escalate the immune response by secretion of yet more inflammatory cytokines. The resulting chain reaction results in ever expanding rounds of inflammatory cytokine secretion, cellular destruction, and yet more cytokine secretion, producing the so-called ‘cytokine storm’, multiple organ failure and even death.

This condition is a significant cause of death in intensive care wards. According to the CDC, at least 1.7 million American adults a year develop sepsis, of whom over 350,000 will die; indeed 1 in 3 people who die in a hospital had sepsis in the course of their hospitalization. Recently WHO has recognized sepsis as a Global Health Priority. (WHA adopts resolution on sepsis. Jena, Germany: Global Sepsis Alliance, 26 May 2017). Other names for this condition are: cytokine release syndrome, macrophage activation syndrome, hemophagocytic lymphohistiocytosis. The initial trigger can be by a variety of stimuli, including, but not limited to: viral infections of the orthomyxoviridae (e.g. influenza) and coronaviridae families (e.g. SARS-COV-2); rheumatic diseases, such as juvenile arthritis and lupus, certain types of blood cancers, like leukemias and lymphomas, the herpes virus family (including those that cause mono and CMV), Ebola, dengue. Most cases triggered by bacterial agents include, but are not limited to, Escherichia coli, Acinetobacter baumanii, Salmonella enterica, Shigella dysenteriae, Pseudomonas aeruginosa, Proteus mirabilis, Serratia marcescens and others) or two- and multiple-celled (Neisseria meningitides, Klebsiella pneumonia, Streptococcus pneumonia, Staphylococcus aureus and others).

Also reported, although less common, are cases of cytokine storm syndrome in patients with rare metabolic disorders and in patients on heart-lung bypass machines such as ECMO. Recently, cytokine storms also have been noted in patients treated with the new chimeric antigen receptor T-cell therapy (CAR-T) as immunotherapy for cancer. In about 20% to 30% of cases, CAR-T saves the patient's life, but it can also trigger uncontrolled inflammation.

Current success in treatment of sepsis and inflammatory cytokine storms are dependent on early diagnosis and intervention and are most effective if they are given before the vicious circle of inflammatory cytokine storms has fully escalated. These methods are primarily focused on combating the primary infection (anti-viral or antibiotic) as well as providing supportive care to treat symptoms such as respiratory insufficiency, heart or kidney failure, maintaining electrolytes and blood pressure. In addition, anti-inflammatory drugs such as corticosteroids or that target cytokines (like siltuximab, tocilizumab and anakinra) may be used. However, there is no drug that can rapidly short-circuit the positive cytokine feedback loop at an advanced stage or return the immune system back to its resting pre-inflammatory state. Hence, the need for novel therapies that work rapidly, effectively, and safely to short-circuit the cytokine positive feedback loop, even when this has reached an advanced state.

Photobiomodulation therapy involves the repeated exposure of the body to therapeutic red or infrared light and has been effectively used for over 100 years in the treatment of many different inflammatory conditions. Photobiomodulation treatment involves a specific intensity, and wavelength of light to which the body is exposed for a defined time interval (typically, 5-30 minutes). The exposure is then repeated at intervals (ranging from 12 hours to every few days) until improvement is noticed. It is safe, has no side effects, and can be targeted to reach specific body tissue or organs. However, results have been contradictory and it has never been successfully used against cytokine storms.

An object of the invention is to provide improved, effective medical apparatus and methods of photobiomodulation therapy.

A further related object of the invention is to provide such improved apparatus and methods as reduce inflammation caused by excessive pro-inflammatory cytokines (cytokine storms) caused by any causative agent.

A further related object of the invention is to to provide such improved apparatus and methods as boost the efficacy and work in synergy with existing pharmaceutical therapies for inflammation, for example, cytokine storms.

### SUMMARY OF THE INVENTION

The inventor has now discovered that the reason for the unreliable effects of prior art treatments have been that PBM light targets complex processes in the cell and therefore treatment must be calibrated to the individual patient for maximum effectiveness. By attention to optimal dosing strategies, the effectiveness of PBM has the potential to be improved by 10-20× in the treatment of deadly cytokine storms, simply by taking an individualized, personalized medicine approach using ML and AI to help define optimal patient treatment.

The foregoing are among the objects attained by the invention, which provides in some aspects an apparatus for treatment of a patient for a life-threatening or other inflammatory condition (e.g., resulting from a “cytokine storm”) that includes a sensor to detect the presence and/or measure (e.g., quantity or quality) of each of one or more biomarkers of the patient, and that generates biomarker data indicative thereof. Those biomarkers include one or more of IL-6, Il-8 and IL-1b; antioxidants Catalase, Glutathione peroxidase 3, Glutathione-Disulfide Reductase; and oxidants Monoamine Oxygenase, NOX1 (NADPH oxidase 1), and COX 4-12 (Cytochrome C oxidase). An illumination source that is in proximity of the patient applies a therapeutic dose of electromagnetic radiation to reduce the inflammation. A controller that is coupled to the sensor and the illumination source generates a dose control signal to effect application of the therapeutic dose by the illumination source. The controller determines an efficacy of applying such a dose by analyzing the biomarker data with an AI/ML module (hereinafter, sometimes referred to as the “AI module,” the “module” or the like)—that is, an artificial intelligence (AI) engine utilizing a machine learning (ML) model—trained with training data indicative of modulation of the one or more biomarkers in each of plurality of members of a subpopulation in response to dosing of such electromagnetic radiation. This can be actual data based on actual measurements of such modulations in individuals to which such doses are applied and/or it can be simulated data based on predictions of such modulations.

Related aspects of the invention, provide an apparatus, e.g., as described above, in which the controller uses a model trained with (i) actual or simulated data indicative of modulation of the patient's (or another individual's) biomarkers in response to dosing of such electromagnetic radiation, in addition to or in lieu of (ii) actual or simulated data indicative of such modulations in a plurality of members of the subpopulation.

Related aspects of the invention provide an apparatus, e.g., as described above, wherein the electromagnetic radiation is infrared light.

Further related aspects of the invention provide an apparatus, e.g., as described above, wherein the illumination source applies the therapeutic dose to the patient in real-time substantially concurrently with detection of the presence and/or measure of the one or more biomarkers of the patient by the sensor.

Still related aspects of the invention provide an apparatus, e.g., as described above, wherein the sensor is a blood or sweat sensor.

Yet related aspects of the invention provide an apparatus, e.g., as described above, wherein the dose control signal designates any of a timing, duration, intensity and wavelength of the therapeutic dose.

Still yet related aspects of the invention provide an apparatus, e.g., as described above, wherein the controller determines successive optimum therapeutic light doses by using the AI engine and ML model to analyze the biomarker data of disease progression together with patient history indications of phenotype, genotype, and/or demographic characterization of the patient.

Yet still related aspects of the invention provide an apparatus, e.g., as described above, for use in connection with concomitant pharmaceutical/nutraceutical treatment of a patient. According to these aspects, the controller modifies the light dose to achieve optimal treatment in the presence of pharmaceutical/nutraceutical agents by analyzing the biomarker data with the AI engine and ML model where that model is trained using biomarker data from individual(s) concomitantly dosed with electromagnetic radiation and pharmaceuticals/nutraceuticals.

Still other aspects of the invention provide methods of training a machine learning model of the type described above for each of a plurality of members of a subpopulation in response to infrared light or other electromagnetic radiation. For each of the plurality of members, the method includes the steps of receiving biomarker data indicative of any of a presence and a measure of each of one or more biomarkers of the member prior to application to that member of electromagnetic radiation; applying electromagnetic radiation to the member, where a timing, duration, intensity and wavelength of the applied radiation is determined by analyzing the received biomarker data with an artificial intelligence (AI) engine utilizing the ML model; identifying a change in the one or more biomarkers subsequent to application of the radiation; and updating the ML model based on the identified change. A related aspect of the invention provides a method of training a machine learning model, e.g., as described above, that additionally includes training the ML model based on physiological responses of the members of the subpopulation to a combination of pharmaceuticals/nutraceuticals and electromagnetic radiation.

A related aspect of the invention provides a method of training a machine learning model, e.g., as described above, that additionally includes training the ML model based on the physiological response of the patient during the course of the treatment. A patient's response is used to adjust dose in the course of each particular treatment.

Related aspects of the invention, provide a method of training an apparatus, e.g., as described above, in which the module is trained at least in part with simulated data indicative of modulation of the one or more biomarkers in each of one or more members of a subpopulation in response to dosing of such electromagnetic radiation. In further related aspects, the module can also be trained by inputting each of a plurality of members of a subpopulation's response to infrared light or other electromagnetic radiation, and with respect to patient history, hospital treatment protocols, etc. The module is also trained in the course of each individual treatments, where it assesses how each successive infrared exposure period modulates multiple biomarkers, which are a measure of the progress of inflammation and also of the underlying cellular metabolic state. In this way the module can adapt to the response of each individual patient during a single treatment.

Still other aspects of the invention provide an apparatus, e.g., as described above, comprising a wearable or otherwise illumination source placed in close proximity to the patient to apply a series of therapeutic doses of electromagnetic radiation which reduce the inflammation. The apparatus includes a sensor that monitors the progression of the disease by detecting the presence and/or measure (e.g., quantity or quality) of each of one or more biomarkers of the patient, and that records biomarker data indicative thereof after each illumination. Those biomarkers include one or more, but are not limited to, inflammatory cytokines IL-6, Il-8 and IL-1b; antioxidants Catalase, Glutathione peroxidase 3, Glutathione-Disulfide Reductase; and oxidants Monoamine Oxygenase, NOX1 (NADPH oxidase 1), and COX 4-12 (Cytochrome C oxidase). The sensor is coupled to an artificial intelligence (AI) engine and a machine learning (ML) module. The ML module is trained with training data indicative of modulation of the one or more biomarkers in each of one or more members of a subpopulation in response to exposure to such electromagnetic radiation. Additional parameters submitted to the AI/ML module include familial background data, patient history, and information on additional adjunctive therapy (pharmaceuticals), such that therapeutic light dose can be adjusted to all the relevant parameters.

In related aspects of the invention, the algorithm uses this information to devise an optimal treatment protocol for the individual patient. Output from the AI/ML module is transmitted to the controller, which initiates the treatment by adjusting the light dose for frequency, timing, intensity, and duration. After the first light exposure, changes in cytokines, oxidants, and anti-oxidant biomarkers are measured in real time to monitor progress of the disease. The data is transmitted by the sensor back to the AI/ML module, which adjusts subsequent light doses according to the response of the patient. Examples may be to shorten or lengthen the interval between light exposures; to shorten or lengthen the light exposure time; or to increase or decrease the light intensity. Other examples, including timing application of the doses in accord with peaks, dips, or other changes in biomarkers during the course of actual treatment. This therapeutic platform provides optimized personalized treatment parameters adjustable throughout the progression of the disease. Still other aspects of the invention provide methods of treatment of a patient for an inflammatory condition paralleling operation of the apparatus described above.

The foregoing and other aspects of the invention are evident in the text that follows and in the drawings.

### BRIEF DESCRIPTION OF THE DRAWINGS

A more complete understanding of the invention may be attained by reference to the drawings, in which:

FIG. 1 depicts hardware components of a photobiomodulation therapy apparatus according to the invention;

FIG. 2 depicts further aspects of the apparatus of FIG. 1;

FIG. 3 depicts a patient-wearable apparatus according to the invention;

FIG. 4 depicts both software and hardware components of an apparatus according to the invention;

FIG. 5 depicts a method of patient therapy according to the invention;

FIG. 6 depicts a method of training a machine learning model according to the invention;

FIG. 7 depicts an apparatus utilizing the method of FIG. 6;

FIG. 8A-8E depict effects on inflammatory biomarkers of varying IR exposure on a population of human cells;

FIGS. 9A-9D depict therapeutic effect of optimised IR exposure therapy in accord with the invention on acute lung inflammation (cytokine storms) in rat animal model;

FIGS. 10A-10B are photographs of lungs of rats following the therapy disclosed in connection with FIG. 9; and

FIGS. 11A-11B are photographs of lungs of rats following the therapy disclosed in connection with FIG. 9.

### DETAILED DESCRIPTION OF THE ILLUSTRATED EMBODIMENT

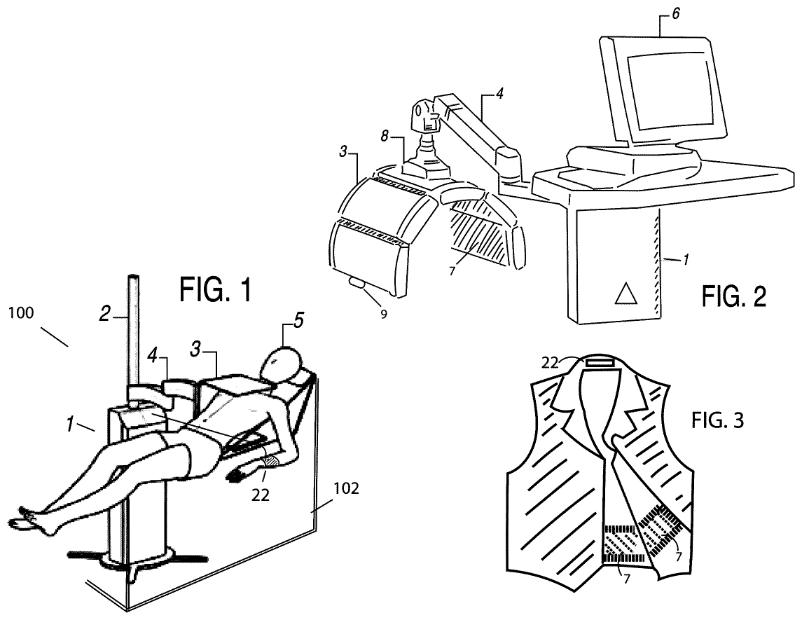

FIGS. 1-2 depict hardware elements of photobiomodulation apparatus 100 for treatment of a patient 5, e.g., for an inflammatory condition, e.g., resulting from a cytokine storm, according to one practice of the invention. Illustrated apparatus 100 includes a base 1 that supports a fixed swing post 2 on which a guide arm 4 is slidably mounted, as shown. A light head 3 is mounted at the distal end of that arm, again, as shown. Base 1, swing post 2 and guide arm 4 are of a conventional construction known in the art suitable for supporting a load—here, light head 3—and to facilitate its being positioned by a caregiver or otherwise for delivering therapy to the patient 5 who in the illustrated embodiment is supported by chair 102 or otherwise, though, who in other embodiments may be ambulatory, all as is within the ken of those skilled in the art in view of the teachings hereof.

Apparatus 100 includes a sensor 22 that detects a presence and/or measure (i.e., quantity or quality) of molecular, histologic, radiographic, physiologic or other biomarkers of the patient. In the illustrated embodiment, that sensor is adapted for detecting biomarkers in the blood, particularly, by way of non-limiting example, one or more of inflammatory cytokines IL-6, Il-8 and IL-1b; antioxidants Catalase (“CAT”), Glutathione peroxidase 3 (“GPX3), Glutathione-Disulfide Reductase (“GSR”); and oxidants Monoamine Oxygenase, NOX1 (NADPH oxidase 1), and COX 4-12 (Cytochrome C oxidase). The sensor may be any sensor suitable for detecting a presence and/or measure of one or more of the aforesaid enumerated sweat-based and/or blood based biomarkers and, preferably (though, not necessarily), a wearable such sensor. Examples include the Sweatronics® for analyte detection from sweat (Eccrine Systems, Cincinnati, Ohio) and the portable Six™ Sensors device technology from Graphene Frontiers inc, Philadelphia, for detecting biomarkers, proteins and amino acids in 5 minute assays. This company has recently expanded into a partnership with Hememics Biotechnology inc. to produce diagnostic chips for real-time analysis of over 100k molecular biologicals (https://www.graphene-info.com/hememics-biotechnologies-and-general-graphene-corporation-form-strategic). The sensor 22 of the illustrated embodiment detects such biomarkers continuously and in real-time, e.g., using nanotechnology, optics or otherwise, though, in other embodiments detection may be periodic or otherwise, all is within the ken of those skilled in the art in view of the teachings hereof.

Illustrated light head 3 emits electromagnetic radiation—e.g., infrared light, preferably, of between 720 nm and 750 nm wavelength—for application to patient 5. Light sources 7 used within the head 3 can be LED panels, incandescent bulbs or otherwise. For example, in the illustrated embodiment, light sources 7 (FIG. 2) used within the head 3 can comprise a high output LED array (720 nm), e.g., of the type commercially available from Quadica Inc., Alberta, Canada or can be custom constructed from commercially available LED chips (eg. from Philips, inc.). Alternatively, or in addition, high output LED infrared floodlights or bulbs ref #N300; #IR720nm AF; #IR720nm AB from Synlyte SAS, Massy Palaiseau, France, can be used, by way of non-limiting example. Moreover, in some embodiments, light sources that output at multiple wavelengths can be employed within the light head, all as is within the ken of those skilled in the art in view of the teachings hereof.

A preferred light head 3 is configured in accord with the shape and size of the portion of the patient's body to be treated. For example, the light head 3 of FIGS. 1-2 is adapted to deliver infrared radiation to the chest of the patient 5 and is, accordingly, C-shaped. The illustrated head 3 is comprised of panels, each comprising or including a light source 7 of the type described above (e.g., an LED panel, incandescent bulb or otherwise), attached by articulating hinges or otherwise to facilitate placing them around and/or conforming them to the patient's chest in a manner that permits uniform delivery of infrared radiation thereto or otherwise. Handles or other structure can be provided to facilitate movement and/or configuration of the light head 3, generally, and/or the light sources therein.

Light head 3 can be fixedly, rotatably, or otherwise coupled to guide arm 4 to further facilitate placing the head 3, generally, and the light sources 7 therein adjacent the region of the patient's body to which infrared radiation is to be delivered. Arm 4 can be articulated or otherwise to facilitate such placement of the head 3 and its light sources vis-a-vis the patient's body.

Of course, the light head 3, generally, and light sources 7 used therein, more specifically, can be configured in other shapes (e.g., flat panels, point sources or otherwise) suitable for delivery of infrared and, more generally, electromagnetic radiation to a desired region of the patient's body, all as is within the ken of those skilled in the art in view of the teachings hereof.

It will be appreciated that the hardware architecture of apparatus 100 illustrated in FIGS. 1-2 and discussed above is merely an example and that the invention may be practiced with other hardware elements, freestanding, wall-mounted, wearable or otherwise, all as is within the ken of those skilled in the art in view of the teachings hereof. Thus, FIG. 3 depicts, by way of example, “hardware” for a wearable photobiomodulation apparatus according to the invention—here, configured as a vest equipped with sensor 22 and light sources 7, only two of which are shown for ease of illustration. The sensor 22 and/or sources 7 may coupled for communication by wire, wirelessly or otherwise to a control unit 20 (discussed below), which may be free-standing, incorporated into the vest itself or otherwise, all as is within the ken of those skilled in the art in view of the teachings hereof.

### Mechanisms

The leading cause of mortality from sepsis caused by viruses or other agents, is exaggerated host immune response triggering cytokine storms, multiple organ failure and death. Current drug-based therapies targeting the pathogen are of limited efficacy once the cytokine storm has initiated. No treatment is reliably effective in the later stages of the disease.

By contrast, photobiomodulation therapy methods and apparatus according to the invention involves brief periods of exposure to red or infrared light, e.g., twice a day or otherwise. It is a non-invasive and safe variation of photobiomodulation therapy. The inventor has devised strategies to use it against hyperinflammatory cytokine storms of the type induced by SAR-COVID2. They showed proof-of-principle that infrared light exposure rapidly and dramatically reduced hyper-inflammation in cultured human alveolar and macrophage cells exposed to SARS-COVID2 viral spike protein. Therefore, controlled periods of infrared light exposure will be used as a therapy for ALI (Acute Lung Inflammation) by way of exampleand can be applied to life-threatening hyper-inflammation triggered by any viral or bacterial elicitor.

Mechanistically, Infrared Light exposure effected by methods and apparatus according to the invention induce an oxidative burst in exposed cells. In cells undergoing inflammation, the small increase in ROS induced by Infrared light triggers massive deregulation of anti-oxidant enzymes. The levels of cellular ROS are rapidly brought down to healthy levels, shutting down the inflammation and short-circuiting the cytokine storm. In cultured human cells, the inventor has seen decrease in inflammatory cytokine secretion by 80% within just a day or two. Therefore, photobiomodulation treatment according to the invention can be effective against cytokine storms, even at late stages where there are very few options.

However, the inventor has also identified the reason that prior art Photobiomodulation therapy has not been proven reliable. Modulation of ROS is a multifactorial process. It depends on a delicate balance between dozens of enzymes and cellular oxidant and antioxidant redox factors, which can vary greatly from individual to individual in quantity and quality, and are also influenced by factors such as heredity, lifestyle, and standard of care being given in the hospital. Likewise, regulation of the cellular immune system involves complex feedback mechanisms involving literally dozens of cellular factors and pathways. In a nutshell, no two people will respond in the identical manner to Photobiomodulation. Optimal timing and intensity of the IR treatments facilitate fighting cytokine storms and even small deviations in treatment regimen can result in 3-5× lower efficiency of treatment.

The primary effect of infrared light is to stimulate mitochondrial metabolic activity and induce transient bursts of ROS (reactive oxygen species). Furthermore, addition of extraneous ROS to cell cultures in the absence of infrared light elicits the same anti-inflammatory effects, indicating that the therapeutic mechanism involves the modulation of ROS via various signaling pathways. ROS homeostasis is a complex process modulated by multiple genetic and environmental inputs in addition to infrared light. Apparatus and methods according to the invention overcome this and make possible photobiomodulation therapy in the treatment of SARS-COVID2 and related viral inflammation.

The inventor has discovered that inflammation triggers (e.g., cytokine secretion) induced in cell cultures by artificial agents as well as by actual viral spike protein can be knocked down by photobiomodulation using infrared light, but that the time to action varies by cell type. For example, the inventor has discovered that knockdown of cytokines with infrared exposure took longer in immune cells than in lung cells. This lead to a realization that the mechanism involved is the photo-stimulation of ROS (reactive oxygen species), the signaling pathways of which vary not only between cell lines but also as result of genetic factors (markers for oxidative stress), environmental factors (e.g., some of the risk factors for COVID), and—for patients already under care—the progression of the treatment.

The inflammatory process treated by methods and apparatus according to the invention is “cell autonomous” in the sense that it occurs within single cells, not requiring complex activation by tissues—hence, treatments effective in cell cultures can be effective in the patient as a whole. Thus, just as with cell cultures, once infrared treatment is started on a patient, he/she will respond by mildly down regulating ROS and then inflammatory cytokines. And, as with cell cultures, one treatment of the patient will not be enough—after a certain time, these levels will come back up—and, if a second treatment is given at a suboptimal time (when cytokines or ROS are still down) it will be far less effective.

This dose can be determined in the actual course of treatment via patient monitoring. Background data on the patient and biomarkers as well as treatment parameter (drugs to be administered, standard of care, etc. are input into an AI/ML module, discussed below. Based on this information, the time and duration of an initial light treatment is determined. The ensuing changes in biomarkers (3 cytokine and 3 ROS) can be measured by the sensors and analyzed. The resulting information is used by the AI module to optimize the second light pulse. The results of this second pulse, input into the AI module, further refines the treatment. Thus personalized doses that will provide the optimal anti-inflammatory therapy will be derived and implemented for each patient. This is contrary to conventional treatment regimens, which are applied on a “one size fits all” basis, which are from 3-6× less effective than an adapted protocol.

### EXAMPLE 1

### Experimental Outline

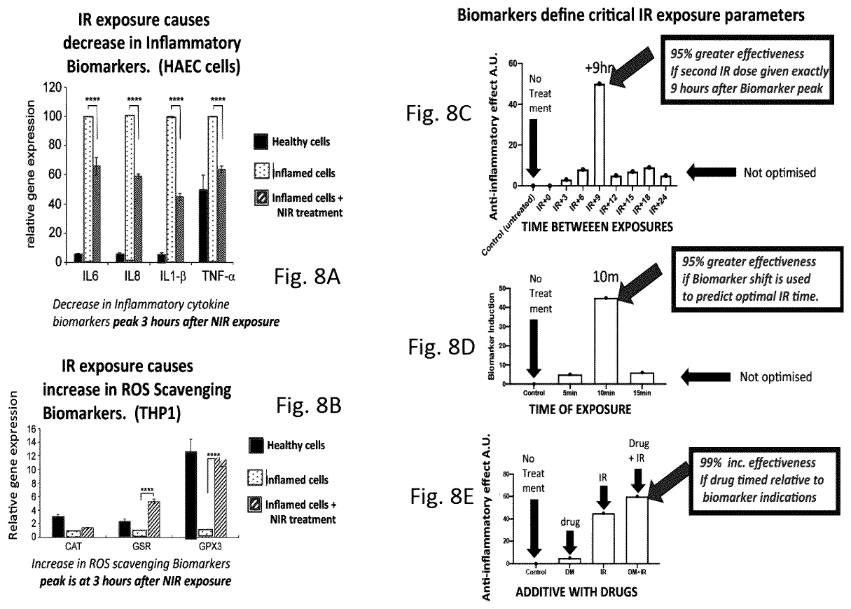

Well-plated samples of Human Type II Alveolar Epithelial Cells (HAEC) isolated from normal human lung tissue, human macrophage THP1 cell lines (AcceGen inc., Fairfield USA) or HEK-Blue™ hTLR4 (Invivogen inc.) were exposed to 100 μM LPS (bacterial lipopolysaccharide inducer) to induce the hyper-inflammatory response in culture. Cells were subsequently exposed to therapeutic NIR illumination at 6 W/m² for the indicated times and intervals as shown (FIG. 8). Synthesis of inflammatory biomarker(s) IL-6, Il-8, IL-1b (FIG. 8a) and of the ROS enzyme biomarkers CAT, GSR and GPX3 and others (FIG. 8B) were measured by qPCR transcriptional gene expression analysis. The ‘anti-inflammatory therapeutic effect’ was the extent to which levels of pro-inflammatory markers Il-6, Il-8, Il-1b and TNFα had decreased in comparison to levels in untreated cells after 48-96 hours of treatment (4-8 exposures in total) (FIG. 8A). In each case the anti-inflammatory effect was compared to that of untreated control cells cultured under the same conditions (As used here, “inflamed” means inflamed cultures that were exposed to LPS but given no NIR treatment). Healthy cell values are also given.

The results (FIG. 8) of the foregoing showed an advantage of precision control over the IR exposure protocol. For a population of HAEC from a single individual, there was a sharp optimum of timing between light treatments, length of light treatment, and dose to achieve synergy with pharmaceutical agents (in this case Dexamethasone, a steroid commonly used as an anti-inflammatory agent). The effectiveness of the treatment dropped by more than 95% when this optimum was not reached. The results showed that this level of precision could be reached when using the relevant biomarkers to monitor the progress of the inflammation and its subsidence (FIG. 8).

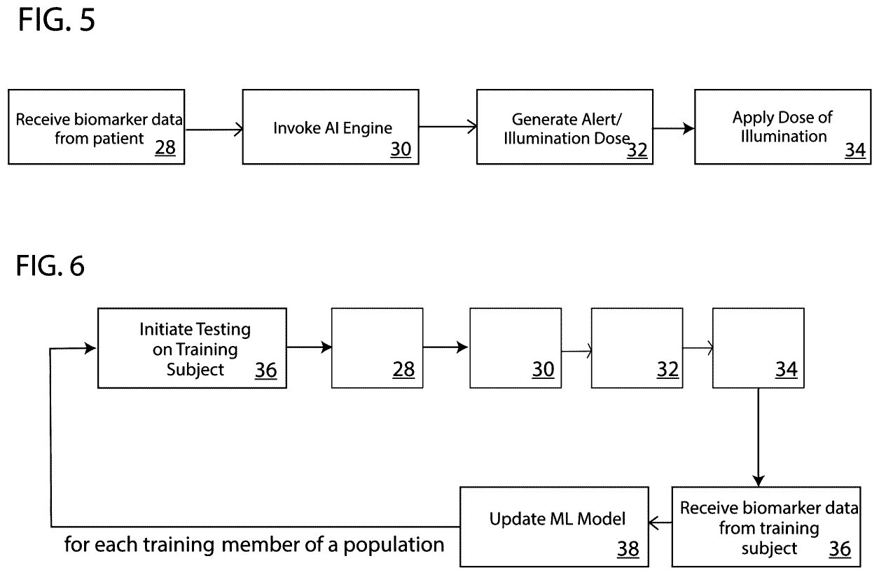

More particularly optimal timing of successive IR pulses of 730 nm, measured at 3 hourly intervals, occurred at 9 hours (and, more precisely, 9 hours +/−0.2 hours, where the latter term reflects the operational timing constraints under which the trial was conducted) after the successive biomarker peaks and, more particularly, of the biomarkers II-6, Il-8, Il-1b and TNFα (FIG. 8 C). The optimal length of exposure was determined from response (or not) of the inflammatory biomarkers (FIG. 8D) to 5 min, 10 min and 15 min exposure durations. These showed that a sharp peak in efficacy occured at 10 minutes (FIG. 8D). Finally, the results showed that DM (dexamethasone), by itself had very moderate anti-inflammatory effects could be combined with IR exposure under the appropriate protocol to yield important synergies (FIG. 8E). More particularly, by way of example, it can be seen that cell cultures induced for inflammation with LPS and treated with 250 nM DM showed little improvement. Multiple 730 nm IR exposure over a 48-hour period resulted in a more significant anti-inflammatory effect, whereas added DM increased this effect such that the combined action of IR and DM reduced inflammation to virtually background levels (FIG. 8E). All these protocols can be further optimized by those skilled in the art without undue experimentation.

Although the example above makes clear that methods according to the invention can achieve treatment of inflammation in cells from multiple individuals, the use of artificial intelligence and machine learning make it possible to optimize treatment of the individuals themselves, albeit, with the specific treatment protocol being determined in accord with the specific response of each such individuals to the antagonist and treatment.

FIG. 8 depicts effects on inflammatory biomarkers of varying IR exposure on a population of human cells in accord with the discussion above. Monitoring of one or more of the selected Biomarkers identified in the drawing can define optimum IR exposure parameters and treatment protocols for methods according to the invention.

Referring to Panels A and B of FIG. 8, inflammation biomarkers are modulated in the course of IR exposure for a population of HAEC from a single individual. Decline in Inflammatory Biomarkers Il-6, Il-8, Il-1b and TNFα (A) and increase in ROS scavenging Biomarkers CAT, GSR and GPX3 (B) can be detected in inflamed model cell lines (Human lung HAEC cell and macrophage THP1 cells) within 3 hours after exposure to infrared light.

Referring to Panel C, biomarker monitoring allows adjustment for optimal timing between successive light pulses. Inflammation induced HEK cell cultures were treated with IR and monitored for modulation of Biomarkers (this occurred in 3 hours in these cultures, as seen in FIG. 8A). Cells were then exposed to additional light pulses timed for 0, 3, 6, 9, 12, 18 or 24 hours after the peak in Biomarker response. There was a sharp response peak at 9 hours (though, the peak and, more particularly, the particulars of response are expected to vary with other biomarkers and other cell lines).

Referring to Panel D, biomarker monitoring provide the optimal exposure time. Inflammation induced HEK cell cultures were treated with IR for 5 min, 10 min and 15 min and monitored for modulation of Biomarkers (this occurred in 3 hours in these cultures, as seen in FIG. 8A). There was a very sharp peak at 10 minutes; other exposure times led to negligible change in biomarkers.

Referring to Panel E, biomarker monitoring allows adjustment of dose to obtain optimal results when adjunct therapies (drugs) are included Inflammation induced HEK cell cultures were treated with IR using an optimized protocol determined as in FIG. 8D. DM was added and showed little effect by itself, whereas when added in synergy with an optimized IR protocol the anti-inflammatory effects were significantly enhanced. This protocol can be further optimized by including relevant biomarkers monitoring the efficacy of DMS throughout progression of the inflammation.

### The Role AI and Machine Learning in Embodiments of the Invention

Further improvements in tailoring treatment protocols of the type described above benefit from the use of machine learning algorithms to model biomarker response in individual and/or groups of patients. Such models can first be trained using simulations, with data in hand, on how biomarkers respond to different light protocols (i.e., variations in timing and length of IR light application) and, later, on how well a specific patient's (or group of patient's) biomarkers respond to the treatment. In this way the best probable dosing regimen can be determined in advance.

Once the treatment of an individual patient has begun, the trained ML model analyses his/her real time response to each successive light exposure. Because treatment can vary from 4-12 light exposures, the ML model can adjust and refine the dosing in each exposure over the entire treatment protocol of 2-6 days. Results data from each individual patient, including data from long-term followup after treatment is incorporated back into the ML model for further refinement. The result is a personalized dose for each patient, according to symptoms, background, hospital practices, and using the optimal palette of biomarkers. An apparatus and method for such treatment is described in further detail below.

### Control Unit 20

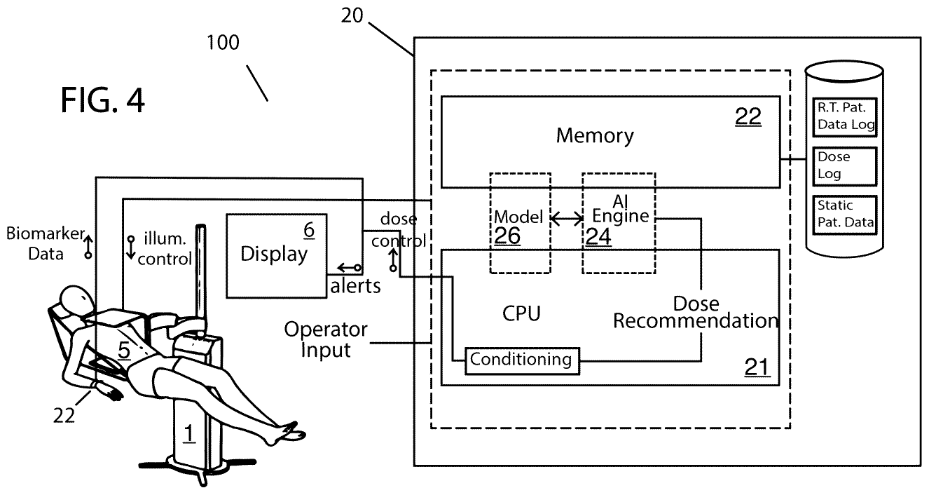

FIG. 4 depicts a control unit 20 of the type used with the apparatus of FIGS. 1-2 to effect the delivery of therapeutic doses of electromagnetic radiation (alternatively, referred to herein as “illumination”) by light sources 7 to patient 5 in response to the presence and/or measure (i.e., quantity or quality) of blood-based or other biomarkers of the patient detected, e.g., by sensor 22. In the embodiment of FIG. 4, the control unit is located in base 1, though, in other embodiments, it may be a stand-alone or co-housed with other apparatus and, indeed, may be remotely disposed from one or more other parts of the apparatus and connected thereto by LAN, WAN, the Internet or otherwise, all as is within the ken of those skilled in the art in view of the teachings herein. As noted above, in the illustrated embodiment dosing is based on detection of one or more of inflammatory biomarker(s) IL-6, Il-8, IL-1b, and of the ROS biomarkers CAT, GSR and GPX3 though, other embodiments may vary in this regard. A number of biomarkers of each category are included, as their relative contribution to the inflammatory pathology may vary in different individuals and under different conditions. By monitoring several enzymes in each category, each of the several distinct relevant cellular pathways leading to the anti-inflammatory response can be independently monitored and the light treatment adjusted so as to achieve an optimal equilibrium.

In some embodiments, the control unit 20 effects delivery of those doses by generating and transmitting to the light sources 7 illumination control signals governing the timing, intensity and duration of electromagnetic radiation to be applied to the patient 5—that is, to be emitted by those light sources for exposure of the patient's body, here, externally via the skin. In embodiments with suitably equipped light sources 7, those control signals can govern the wavelength of the illumination doses instead or in addition, e.g., whether wide spectrum (by incandescent-type sources 7), narrow spectrum (by LED-type sources 7), of varied wavelength or otherwise, all as is within the ken of those skilled in the art in view of the teachings hereof. In other embodiments, the control unit 20 effects delivery of those doses by generating and transmitting to display 6 an alert that such a dose is to be delivered by actuation of the light sources, e.g., by a caregiver, and at what timing, intensity, duration and/or wavelength. The control unit of still other embodiments effects dose delivery by generating and transmitting both illumination control signals and alerts to the light sources 7 and display 6, respectively. In the discussion that follows the term “dose control” signals is used in reference to both illumination control signals and alerts, the applicability of one or both of which in any given instantiation is implementation-dependent.

Control unit 20 of the illustrated embodiment is a digital data processor 21, instructions and data for execution by which are maintained in memory 22, which may comprise any or a combination of random-access memory (RAM), flash memory, solid state or inertial disks, or otherwise, all per convention in the art as adapted in accord with the teachings hereof. This can include, by way of non-limiting example, instructions (and data) comprising an AI engine 24 and machine learning model 26, which together analyze biomarker data received from sensor 22, caregiver/operator input (e.g., via keyboard, touchscreen or otherwise (not shown)) or otherwise indicative of a presence and/or measure of the aforesaid blood-based or other biomarkers of the patient 5 and effect generation (and transmittal) of dose control signals as discussed below and elsewhere herein. Storage of such instructions within memory 22 and their transfer to and use by the processor 21 for the foregoing purposes is within the ken of those skilled in the art in view of the teachings hereof.

In some embodiments, the engine 24 and model 26 are independent, interoperable components within control unit 20. In other embodiments, the engine and model (whether or not identified as such) are integrally combined with one another, in whole or in part, to define an ML-trained and/or ML-trainable artificial intelligence. Thus, the illustration of and reference to the AI engine 24 and model 26 as independent units, as immediately above and elsewhere herein, is without loss of generality and includes integral combinations of those units, in whole or in part.

Data stored in memory 22 can include real-time patient data (e.g., a log of biomarker data received from sensor 22 regarding patient 5), static patient data (e.g., phenotype, genotype, demographic and/or other “static” data characterizing the patient 5) and a log of values of dose control signals generated and transmitted by the control unit. This data may be stored within memory and/or in supplemental storage, whether onboard the control unit 20, remotely-stored or otherwise.

### Illumination Dose Determination

FIG. 5 depicts steps executed by control unit 20 to determine an illumination dose recommendation that determines the value(s) contained in the dose control signal. More particularly, that drawing depicts steps executed by that unit 20 in use of the artificial intelligence (AI) engine 24 and machine learning (ML) model 26 to automatically analyze biomarker data received from the sensor 22—and, optionally, static patient data as discussed above—to predict when it would be efficacious to deliver a dose of electromagnetic radiation to the patient 5 and, if so, the intensity, duration and/or wavelength of that dose.

In step 28, the control unit 20 receives the biomarker data in real-time from sensor 22, i.e., substantially concurrently with detection of the corresponding biomarker(s) in the patient 5. In some embodiments, that data is not received by unit 20 directly from the sensor but, rather, is entered in whole or part by the caregiver or other operator via a keyboard, touchpad or otherwise. At the same time, the control unit 20 can retrieve from memory 22 or otherwise static patient data, if any, that will be used by the AI engine 24 and model 26, along with the biomarker data, to generate an illumination dose recommendation. Alternatively, or in addition, such static data can be accepted via operator input and/or from read-only (or other) memory on-board the sensor, all by way of non-limiting example.

In step 30, the control unit 20 invokes the AI engine 24 to generate a dosing recommendation from the biomarker data (and, in some implementations, additionally from the static data for patient 5) using the machine learning (ML) model 26. In some embodiments, that dosing recommendation is based on detection of peaks, dips, or other changes in the measured biomarker values and, more particularly, our way of non-limiting example, can be based on peaks in the measured value of biomarkers X, Y and/or Z. Thus, in accord with the example discussed below, the dosing recommendation can be generated to effect dosing of the patient a designated period of time after a peak (or other change) in one or more of those biomarkers.

In step 32, the control unit 20 generates a dose control signal based on the dosing recommendation in step 30. That dose control signal can be identical to the dosing recommendation, though more typically, the dose control signal is adapted, in the case of illumination control signals to include formatting, scaling and other “conditioning” to meet protocol requirements of the light sources and, likewise, in the case of alerts, to include (in addition to formatting) verbiage suitable for display to the caregiver. In embodiments that operate in real-time, the dose control signal (i.e., the illumination control signal and/or alert) can be generated concurrently with the recommended timing of the dose to effect substantially immediate dosing. Alternatively and/or in addition, the dose control signal can contain timing information to effect delivery at a later time or times. Implementation of logic contained in the control unit, the light head 3, the light sources 7 or otherwise to actuate the light sources in accord with such dose control signals (and, more specifically, illumination control signals) is within the ken of those skilled in the art in view of the teachings hereof.

In step 34, the light source(s) 7 are actuated—as a result of application of the illumination control signal and/or action of the caregiver in response to the alert—and the recommended dose of infrared or other electromagnetic radiation illumination is applied to patient 5.

Returning to discussion of step 30, the ML model 26 provides parameters (e.g., weights and biases) for use by the AI engine 24 in making the dosing recommendation per convention in the art as adapted in accord with the teachings hereof. Those parameters are established during training of the model 26 with training biomarker data, which can be generated from human subjects or from epithelial, embryonic or other cell cultures-in the latter case, with suitable sensors 22′ (used alone and/or in combination with reagents, luminescent tags or otherwise) for detecting the presence and/or quantities of biomarkers produced by the cells in culture plates 40 disposed on conveyor 42 or otherwise, implementation of which is within the ken of those skilled in the art in view of the teachings hereof.

Whereas the model 26 can be trained in connection with execution of steps 28-32, i.e., while generating and applying dosings to patient 5, creation and training of the model 26 typically occurs beforehand, during a training phase, that is typically (though not necessarily) effected on a separate “training” digital data processor (not shown) that is constructed and operated similarly to control unit 20 but that operates independently of that unit.

It will be appreciated that, whereas the AI engine 26 is utilized within controller 20 during patient treatment to generate dosing recommendations using the model 26, the same engine 24 is used during the training phase to train that model 26 (e.g., before its subsequent download to controller 20 for runtime use in treating patients 5). And, while a single common AI engine 24 and ML model 26 can be concurrently shared between the controller 20 and the training digital data processor, as a practical matter, separate instantiations of that engine 24 and model 26 are copied between the training digital data processor and controller 20 for runtime use (i.e., while treating patients). While those copies can be functionally identical, that maintained on controller 20 can be adapted for mission-critical field use and need not include a training mode, while that on training digital data processor can be adapted for model training.

Although potentially presenting regulatory hurdles, in some embodiments, training and runtime phases can be combined in a single device, e.g., as where a caregiver or other operator of apparatus 100 places the onboard AI engine 24 in training mode following a patient treatment session and inputs to it further biomarker data from the patient 5, which the engine 24 then feeds-back to the model 26 for updating per convention in the art as adapted in accord with the teachings hereof.

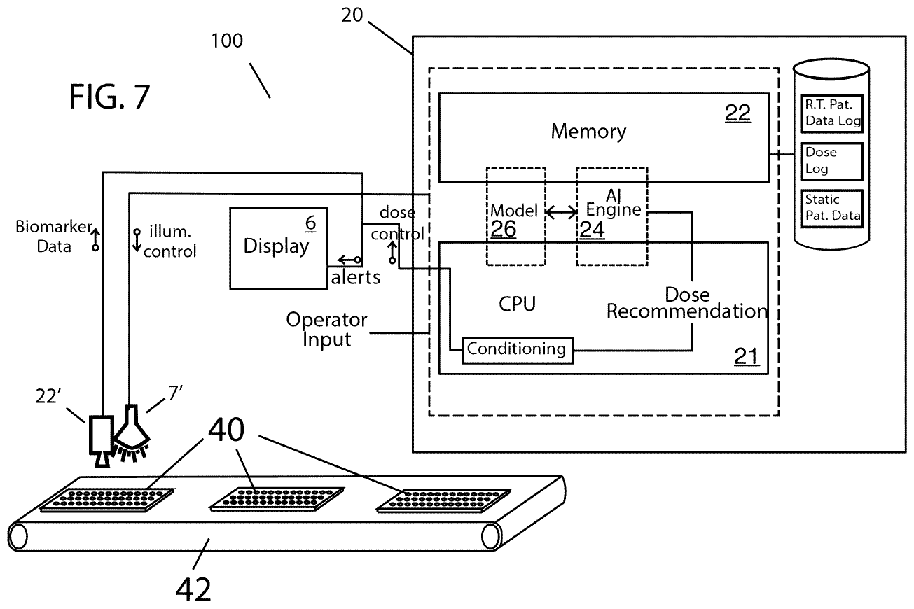

FIGS. 6-7 depict an apparatus and method for training the ML model 26 according to one practice of the invention. In step 36, a cell sample extracted from a training subject who can be an expected patient or, for example, more likely, a member of a subpopulation of individuals having a genotype, phenotype, demographic or other characteristics in common with an expected patient 5 is deposited in a well plate 40 and positioned adjacent light source 7′ (which simulates electromagnetic radiation emitted by light source 7 of apparatus 100) and sensor 22′ (discussed above). Biomarker data is collected from the sample and a dose of illumination is applied to it in accord with the method discussed above in connection with steps 28-34. In step 36, the biomarker data is collected from the cell sample at one or more times subsequent to treatment (e.g., at 1-minute, 5-minute, 30-minute, 1-hour, 4-hour and/or other suitable intervals). That follow-up data indicates whether or not and to what degree the dosing improved (or worsened) the training sample's condition (e.g., vis-a-vis production of inflammation triggers) as reflected by the biomarkers. In step 38, the ML model 26 is updated based on those biomarker values, all per convention in the art of training ML models as adapted in accord with the teachings hereof.

The foregoing can be repeated with samples from each of plural members of the subject subpopulation. Preferably, those samples are from members of a subpopulation as described above, though, that is not a requirement of the invention. And, though FIGS. 6-7, depicts training using samples extracted from test subjects, in some embodiments training is performed using the patients themselves as subjects on apparatus 100, e.g., of FIGS. 1-2.

Although in the illustrated embodiment, the ML model 26 is trained using actual data, collected as shown in FIGS. 6-7 and described above, in other embodiments, that model is trained, at least in part, using simulated data, that is, data reflecting predicted or expected changes in biomarker data of a patient, another individual, and/or a subpopulation in response to application of electromagnetic radiation as discussed above. It will thus be appreciated that, depending on the embodiment, training data for the model 26 can be actual data, simulated data, or a combination of both.

In some embodiments, the ML model 26 is trained in particular regard to the effect on the training sample's production of inflammation triggers (e.g., as reflected by peaks, dips or other changes in specific biomarkers) vis-à-vis timing of radiation dosing. Such models can be used beneficially with treatment apparatus of the type described above in which dosing recommendations are based on detection of peaks, dips, or other changes in the measured biomarker values and, more particularly, by way of non-limiting example, with treatment apparatus that effect dosing of the patient had a certain time interval after a peak, dip or other change in one or more of those biomarkers.

### Recommended Pharmaceutical/Nutraceutical Dose Determination

Described above are apparatus and methods for delivering doses of electromagnetic radiation, e.g., infrared light, to a patient 5 based on recommendations of AI engine 24 and ML model 26, trained as described above. In some embodiments, the AI engine 25 and model 26 additionally deliver dosing recommendations for pharmaceuticals and/or nutraceuticals to be injected, ingested or otherwise given to the patient 5 in connection with the infrared light dosings. Those embodiments are architected and operated identically to the embodiments shown in FIGS. 1-7 and described above, although, (i) in addition to generating a dose control signal for triggering illumination by light sources 7 in step 32, in these embodiments a dose control signal in the form of an alert (for presentation on display 6) is generated directing the caregiver on the timing and quantity of pharmaceutical/nutraceutical to be given to the patient 5, e.g., in connection with radiation dosing, and (ii) in addition to sensing biomarkers, the controller 20 can accept from a caregiver or other operator, from a store of static data associated with the patient 5 or otherwise information on the status of the patient's current pharmaceutical/nutraceutical treatment regimen. These same additional inputs and outputs can be used/generated during the training in order to train model 26, all as is within the ken of those skilled in the art in view of the teachings hereof.

### EXAMPLE 2

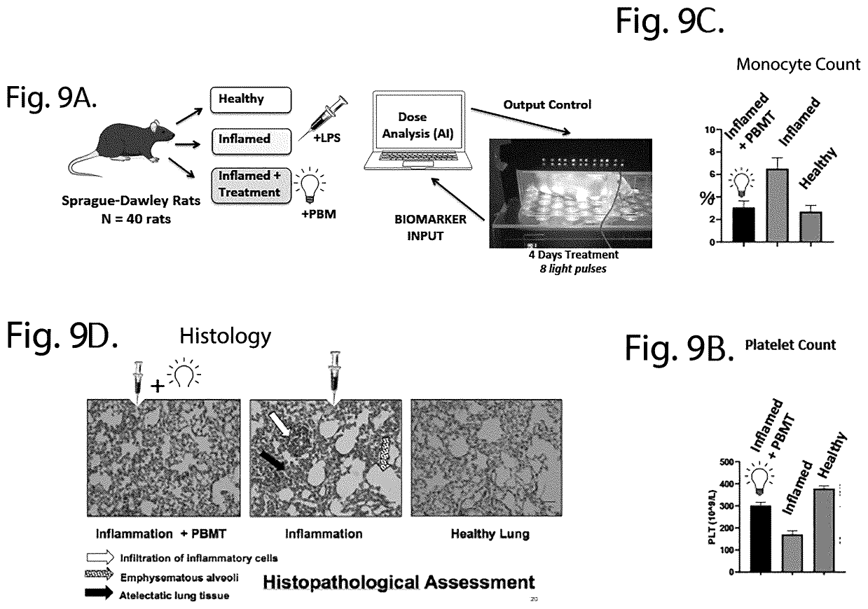

Photobiomodulation therapy (PBMT) in accord with the invention was performed on male Sprague-Dawley rats in which ALI (acute lung inflammation) had been induced by inhalation of a bacterial elicitor known as LPS (lipopolysaccharide, purchased from SIGMA, St. Louis Mo) at 10 mg/Kg body weight at Day 0, followed by boosters of 10 mg/Kg and 7 mg/Kg at days 1 and 2, respectively. (FIG. 9). This bacterial elicitor causes the same pathology by the same underlying mechanism as does infection with SARS-COVID2 in humans, namely by causing hyperinflammatory, disregulated cytokine storms in the lungs. This method is used interchangeably with viral induction as a model of acute lung inflammation caused by COVID.

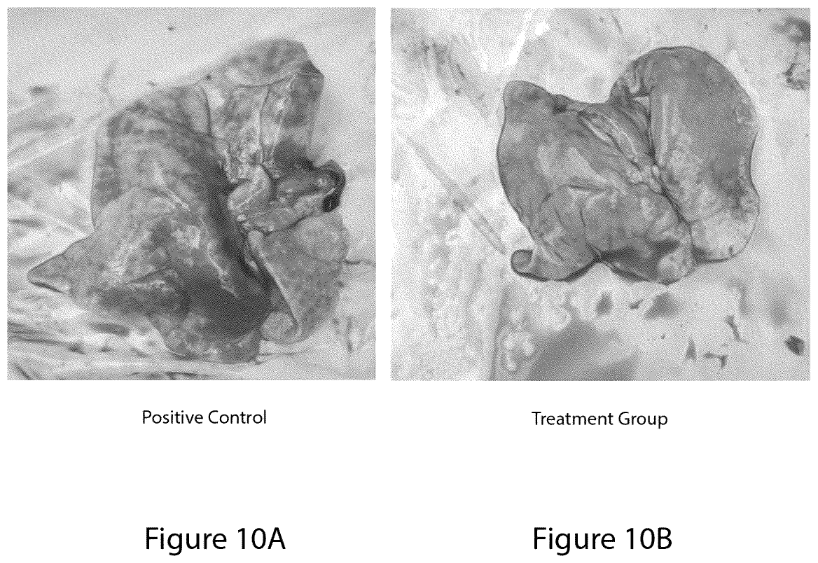

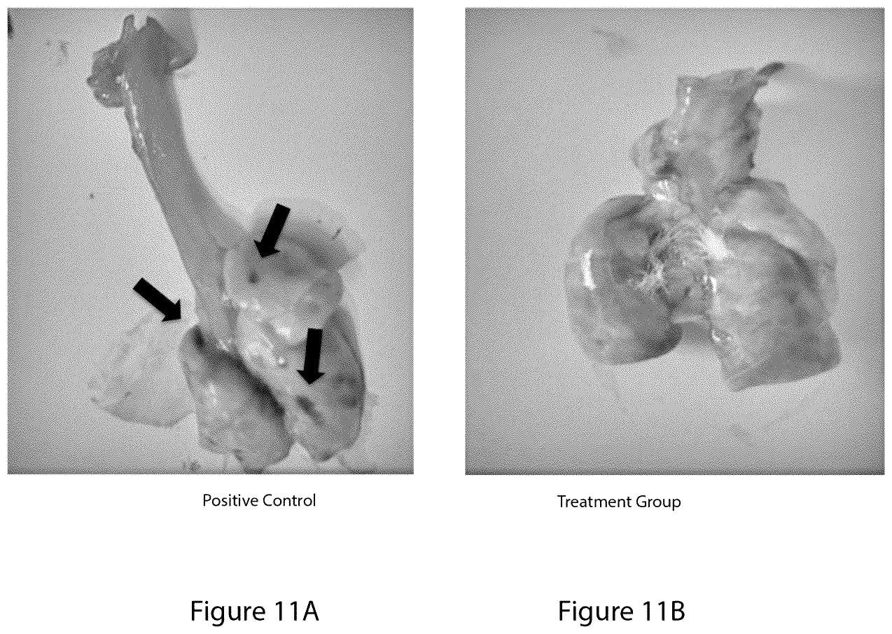

In our trial, rats used were aged 8-16 weeks, between 200-400 g, with no sign of respiratory distress or other systemic illness and normal behavioural and feeding patterns. Anesthetised animals were induced for ALI through inhalation of LPS on consecutive days and treated with 8 (eight) short exposures to defined Infrared (IR) light adjusted according to the response parameters of some known biomarkers for reactive oxygen species (see FIG. 8B, FIG. 9A). Positive control rats were induced to develop acute lung inflammation (ALI), but without exposure to IR light (FIG. 9A). After 4 days of treatment, rats were sacrificed and the progress of pulmonary inflammation monitored by markers of inflammation including platelets (FIG. 9B) and neutrophil (FIG. 9C) concentration in the blood in treated and untreated groups. Histpathological analysis was particularly striking (FIG. 9D). The positive control (inflamed) group showed severe emphysematous alveoli as well as collapse (atelectasis), indicating a heterogeneous pattern of lung damage with multifocal areas of mononuclear inflammatory cells, interlobar septa (thickened tissue separating lung lobes), and infiltration of inflammatory cells, indicative of severe inflammation. All of these markers were virtually absent in the treatment group. Additional measured parameters included levels of pulmonary edema, cytokines, lung tissue wet/dry ratio, X-ray data and so on (not shown). Progress of the pulmonary inflammation was also observed visually, through dissection of the rat lungs. Two representative sets of lungs are shown in FIGS. 10A and 10B (comparison of positive control group and the treatment group which had received 4 days of PBMT exposure). As evident in these photographs, the lungs of the control group show multiple surface lesions and hemmorhaging, whereas those of the treatment group showed smooth morphology similar to healthy control rats. Using this approach we were able to achieve almost full recovery (80% or more recovery from ALI symptoms in the treated rats as compared untreated controls as determined by decrease in cytokine levels, blood platelet and neutrophil concentrations to levels approaching those in healthy rats (see FIG. 9B, C, and Table 1)]. This improvement was achieved in just 4 days of treatment. A table reflecting the trial is provided below.

TABLE 1
Markers for Inflammation in Rats induced
for Acute Lung Inflammation

| | Negative | Positive | Treatment |
|---|---|---|---|
| Markers for | Control | Control | Group |
| Inflammation | (Healthy) | (+LPS) | (+LPS + PBM) |
| Serum Il-6 (pg/ml) | 3766 +/− 240 | 5100 +/− 350 | 3266 +/− 300 |
| Monocytes (%) | 2.7 +/− .4 | 6.52 +/− .5 | 3.0 +/− .3 |
| Neutrophils (%) | 35.5 +/− 3 | 58.6 +/− 4 | 43.3 +/− 3 |
| Platelet Count | 379 +/− 41 | 171 +/− 20 | 302 +/− 35 |
| (×10⁹ per Liter) | | | |
| White Blood Cells | 10.23 +/− .4 | 18.7 +/− .3 | 14.4 +/− .25 |
| (×10⁹ per Liter) | | | |
| Lung Wet/Dry ratio | | 4.4 +/− .39 | 3.47 +/− .25 |
| (measure of Edema) | | | |

Explanation: Rats were induced for Acute Lung Inflammation through inhalation of bacterial LPS elicitor and treated with Photobiomodulation therapy as described in FIG. 9. Markers for inflammation were compared in treated, untreated, and healthy control rats.

We conclude that, given further optimization of the treatment dose by an AI driven dosing system, an even faster and more complete recovery should be achievable, based on in vitro data from our cell culture model recovery occurs in some cases within 48 hours of treatment (FIG. 8)

FIG. 9: Proof-of-principle RAT animal trials applying Photobiomodulation therapy for Acute Lung Inflammation caused by SARS-COVID2.

Referring to Panel A, matched animals were divided into 3 groups which included healthy animals, animals exposed to LPS to induce Acute Lung Inflammation, and animals exposed to LPS and treated with photobiomodulation therapy (PBMT). The dose was established from preliminary results monitoring production of ROS as a function of exposure time and interval. All animals in the treatment group were exposed to IR light at 12 hour intervals over a four day period, and all animals were sacrificed and the progress of the inflammatory response was measured on the fifth day.

Referring to Panel B, as markers for inflammation, platelet count (per ml whole blood) was measured and shown to be markedly reduced in the inflamed group (middle bar) as compared to the healthy control group (right bar). However, these levels recovered to within 20% of healthy controls in the PBMT treatment group (left bar).

Referring to Panel C, the percentage of monocytes was significantly increased in the inflamed group (middle bar) as compared to the healthy uninflamed control group (right bar). PBMT therapy restored levels of monocytes to those of the healthy controls (left bar). Panel D. Histochemical sections of lung tissue from LPS induced animals (inflamed) showed severe thickening of blood vessel walls, infiltration of inflammatory cells (green arrow), emphysematous alveoli (yellow arrow),and atelectatic lung tissue (white arrow) indicative of severe inflammation (centre panel). All of these parameters were significantly recovered in lung cells from animals treated with PBMT (left panel), which approached those of the healthy animals (right panel).

The above results can be improved (e.g., greater recovery in shorter time) in embodiments in which the above data is used to train an ML model, as discussed above, in order to permit tailored therapy based on individual patent response.

### Conclusion

Described above are apparatus and methods meeting the objects set forth herein. It will be appreciated that the embodiments illustrated in the drawings and detailed above are merely examples of the invention and that other embodiments varying therefrom fall within the scope of the invention, of which I claim:

1. Apparatus for treatment of a patient for an inflammatory condition, comprising:
A. a sensor that detects any of a presence and a measure of each of one or more biomarkers of the patient, and that generates biomarker data indicative thereof, where the biomarkers include one or more of IL-6, Il-8 and IL-1b; antioxidants Catalase, Glutathione peroxidase 3, Glutathione-Disulfide Reductase; and oxidants Monoamine Oxygenase, NOX1 (NADPH oxidase 1), and COX 4-12 (Cytochrome C oxidase),
B. an illumination source in proximity of the patient that applies a therapeutic dose of electromagnetic radiation thereto, and
C. a controller in communications coupling with the sensor and with the illumination source, the controller generating a dose control signal to effect application of the therapeutic dose by the illumination source, the controller determining an efficacy of applying such a dose by analyzing the biomarker data with an artificial intelligence (AI) engine and a machine learning (ML) model trained with training data indicative of modulation of the one or more biomarkers in any of (i) each of plurality of members of a subpopulation in response to dosing of such electromagnetic radiation, (ii) the patient or another individual in response to such dosing.

2. The apparatus of claim 1, wherein
the electromagnetic radiation is infrared light, and
the controller generates the dose control signal to effect application of the therapeutic dose timed as a function of peaks, dips or other changes in the measures of one or more biomarkers.

3. The apparatus of claim 1, wherein the illumination source applies the therapeutic dose to the patient in real-time substantially concurrently with detection of the presence and/or measure of the one or more biomarkers of the patient by the sensor.

4. The apparatus of claim 1, wherein the sensor is a blood sensor.

5. The apparatus of claim 1, wherein the dose control signal represents any of a timing, duration, intensity and wavelength of the therapeutic dose.

6. The apparatus of claim 1, wherein the controller determines an efficacy of applying a said therapeutic dose by analyzing the biomarker data along with an indication of a phenotype, genotype, and/or demographic characterization of the patient with a said artificial intelligence (AI) engine and a said machine learning (ML) model trained with training data indicative of modulation of the one or more biomarkers in each of plurality of members of a subpopulation of at least comparable phenotype, genotype, and/or demographic characterization in response to dosing of such electromagnetic radiation.

7. The apparatus of claim 1, wherein the controller generates one or more dose control signals to effect application of a recommended therapeutic dose to the patient of a pharmaceutical/nutraceutical by analyzing the biomarker data with a said artificial intelligence (AI) engine and a said machine learning (ML) model trained with training data indicative of modulation of the one or more biomarkers in each of plurality of members of a subpopulation in response to dosing of each of electromagnetic radiation and such pharmaceutical/nutraceutical.

8. The apparatus of claim 7, wherein the pharmaceutical/nutraceutical as any of a steroid and a monoclonal antibody.

9. A method of generating electromagnetic radiation, comprising training a machine learning (ML) model by
A. determining a modulation of one or more of biomarkers in each of plurality of members of a subpopulation in response to infrared light by, for each of the plurality members,
B. receiving biomarker data indicative of any of a presence and a measure of each of one or more biomarkers of the member prior to application to that member of infrared light,
C. applying infrared light to the member, where a timing, duration, intensity and wavelength of the applied infrared light is determined by analyzing the received biomarker data with an artificial intelligence (AI) engine utilizing the ML model,
D. identifying a change in the one or more of the biomarkers subsequent to application of the infrared light, and
E. updating the ML model based on the identified change.

10. The method of claim 9, wherein the training step comprises training the machine learning (ML) model
A. by determining a modulation of the one or more of biomarkers in each of the plurality of members of the subpopulation in response to infrared light and one or more pharmaceuticals/nutraceuticals by, for each of the plurality members,
B. receiving biomarker data indicative of any of a presence and a measure of each of one or more biomarkers of the member prior to application to that member of infrared light and the one or more pharmaceuticals/nutraceuticals,
C. applying infrared light and the one or more pharmaceuticals/nutraceuticals to the member, where a dose of the applied pharmaceuticals/nutraceuticals and a timing, duration, intensity and wavelength of the applied infrared light is determined by analyzing the received biomarker data with an artificial intelligence (AI) engine utilizing the ML model,
D. identifying a change in the one or more of the biomarkers subsequent to application of the infrared light and the one or more pharmaceuticals/nutraceuticals, and
E. updating the ML model based on the identified change.

11. An automated method of treatment of a patient for an inflammatory condition, comprising:
A. receiving biomarker data indicative of any of a presence and a measure of each of one or more biomarkers of the patient,
B. generating a dose control signal to effect application of a recommended therapeutic dose of electromagnetic radiation to the patient by determining an efficacy of applying such a dose by analyzing the biomarker data with an artificial intelligence (AI) engine and a machine learning (ML) model trained with training data indicative of modulation of the one or more biomarkers in any of each of (i) plurality of members of a subpopulation in response to dosing of such electromagnetic radiation, (ii) the patient or another individual in response to such dosing,
C. where the one or more biomarkers include one or more of IL-6, Il-8 and IL-1b; antioxidants Catalase, Glutathione peroxidase 3, Glutathione-Disulfide Reductase; and oxidants Monoamine Oxygenase, NOX1 (NADPH oxidase 1), and COX 4-12 (Cytochrome C oxidase).

12. The method of claim 11, comprising applying therapeutic dose to the patient in accord with the dose control signal.

13. The method of claim 1, wherein the electromagnetic radiation is infrared light and the controller generates the dose control signal to effect application of the therapeutic dose timed as a function of peaks, dips or other changes in the measures of one or more biomarkers.

14. The method of claim 1, wherein step (A) comprises receiving the biomarker data in real time.

15. The method of claim 14, comprising receiving the biomarker data from measurements based on the patient's blood.

16. The method of claim 11, wherein step (B) comprises generating the dose control signal to effect application of the therapeutic dose in real-time.

17. The method of claim 11, wherein the dose control signal represents any of a timing, duration, intensity and wavelength the therapeutic dose.

18. The method of claim 11, wherein the determining step includes determining an efficacy of applying a said therapeutic dose by analyzing the biomarker data along with an indication of a phenotype, genotype, and/or demographic characterization of the patient of the patient with a said artificial intelligence (AI) engine and a said machine learning (ML) model trained with training data indicative of modulation of the one or more biomarkers in each of plurality of members of a subpopulation of at least comparable phenotype, genotype, and/or demographic characterization in response to dosing of such electromagnetic radiation.

19. The method of claim 11, comprising generating one or more dose control signals to effect application of a recommended therapeutic dose to the patient of a pharmaceutical/nutraceutical by analyzing the biomarker data with a said artificial intelligence (AI) engine and a said machine learning (ML) model trained with training data indicative of modulation of the one or more biomarkers in each of plurality of members of a subpopulation in response to dosing of each of electromagnetic radiation and such pharmaceutical/nutraceutical.

20. The method of claim 19, wherein the pharmaceutical/nutraceutical as any of a steroid and a monoclonal antibody.
